(19) United States
(12) Patent Application Publication
(10) Pub. No.: US 2016/0317696 A1
(43) Pub. Date: Nov. 3, 2016

(54) A Process for Making a Hydrophilic Nonwoven Structure, a Nonwoven Structure Produced Thereby and an Article Containing the Nonwoven Structure

(71) Applicant: DOW GLOBAL TECHNOLOGIES LLC, Midland, MI (US)

(72) Inventors: Rudolph J. Koopmans, Einsiedeln (CH); Evren Aslan-Guerel, Zuerich (CH); Gert J. Claasen, Richterswil (CH)

(21) Appl. No.: 15/108,310

(22) PCT Filed: Dec. 15, 2014
(86) PCT No.: PCT/US2014/070258

§ 371 Date: Jun. 26, 2016

Related U.S. Application Data
(60) Provisional application No. 61/922,512, filed on Dec. 31, 2013.
(60) Provisional application No. 62/040,653, filed on Aug. 22, 2014.

(51) Int. Cl. A61L 15/42; D06M 10/02; A61L 15/22; A61F 13/15; A61F 13/51

(52) U.S. Cl. CPC A61L 15/42; A61F 13/15731; A61F 13/15658; A61F 13/51; A61L 15/225; D06M 10/02; A61F 2013/51069; A61F 2013/51023

(57) ABSTRACT

A process for making a hydrophilic nonwoven structure comprising: forming a nonwoven structure comprising fibers; and exposing the nonwoven structure to an atmospheric plasma comprising an inert gas and a substance having a polar group and which can be vaporized or made into an aerosol and which forms a free radical upon exposure to a dielectric barrier discharge is provided. Also provided are nonwoven structures produced thereby and articles containing the nonwoven structures.

### FIELD

The present disclosure relates to a process for making a hydrophilic nonwoven structure, a nonwoven structure produced thereby and an article containing the nonwoven structure.

### BACKGROUND

Typically non-woven based hygiene products are composed of different layers in which one or more layers are made of non-polar polyolefin plastics such as polyethylene (PE) and polypropylene (PP). Such materials may be used in personal hygiene type articles which include a topsheet, which are placed adjacent to the body of the wearer, a backsheet placed away from the body of the wearer, and a core for collecting and holding bodily fluids disposed between the topsheet and backsheet. The polyolefin based nonwoven materials are commonly used as topsheet or core material to collect and keep bodily fluids in the hygiene product. For comfort, such hygienic articles should have hydrophilic top sheets, which are placed adjacent to the body, and distribution layers.

Current solutions for improving the hydrophilicity of nonwoven materials result in materials which show a decrease in hydrophilicity following repeated exposures to liquids, such as saline solutions mimicking bodily fluids. A hydrophilic nonwoven material which maintains hydrophilicity following repeated fluid exposure and a method of making the same would be beneficial. It would be further beneficial to have a method useful in continuous in-line or semi continuous production environment.

### SUMMARY

Disclosed in embodiments herein are processes for making a hydrophilic nonwoven structure, nonwoven structures, and articles containing nonwoven structures.

In one or more embodiments, the present disclosure provides a process for making a hydrophilic nonwoven structure comprising forming a nonwoven structure comprising fibers; and exposing the nonwoven structure to an atmospheric plasma comprising an inert gas and a substance having a polar group and which can be vaporized or made into an aerosol and which forms a free radical upon exposure to a dielectric barrier discharge.

In one or more embodiments, the present disclosure provides a nonwoven structure.

In one or more embodiments, the present disclosure provides an article comprising one or more embodiments of the nonwoven structure disclosed herein.

### BRIEF DESCRIPTION OF THE DRAWINGS

For the purpose of illustrating the disclosed subject matter, there is shown in the drawings a form that is exemplary; it being understood, however, that the present disclosure is not limited to the precise arrangements and instrumentalities shown.

FIG. 1 is a graph illustrating the contact angle of a saline drop versus time for each of Inventive Examples 4-6 and Comparative Example 1, wherein the filled triangles correspond to Inventive Example 4, the open squares correspond to Inventive Example 5, the open diamonds correspond to Inventive Example 3 and the solid squares correspond to Comparative Example 1;

FIG. 2 is a graph illustrating the contact angle of a saline drop versus time for each of Inventive Examples 4-6, tested 6 weeks after the plasma treatment, wherein the filled triangles correspond to Inventive Example 4, the solid squares correspond to Inventive Example 5, the open diamonds correspond to Inventive Example 3;

FIG. 3 is a graph illustrating the contact angle of a saline drop versus time for Inventive Example 4, for a first insult and a second insult of the saline solution;

FIG. 4 is a graph illustrating the XPS surface composition of Inventive Example 4, before and after atmospheric plasma treatment; and

FIG. 5 is a schematic illustrating the equipment used to measure contact angle.

### DETAILED DESCRIPTION

The present disclosure is a process for making a hydrophilic nonwoven structure, a nonwoven structure produced thereby and an article containing the nonwoven structure.

The process for making a hydrophilic nonwoven structure comprises forming a nonwoven structure comprising fibers; and exposing the nonwoven structure to an atmospheric plasma comprising an inert gas and a substance having a polar group and which can be vaporized or made into an aerosol and which forms a free radical upon exposure to a dielectric barrier discharge. Atmospheric plasma systems and methods are generally described in U.S. Pat. No. 5,433,786, the disclosure of which is incorporated herein by reference.

In an alternative embodiment, the present disclosure further provides a hydrophilic nonwoven structure produced by the process according to any embodiment disclosed herein.

In another alternative embodiment, the present disclosure further provides a nonwoven structure comprising fibers having a chemically modified surface, wherein the chemically modified surface comprises a hydrophilic moiety covalently bonded to a polymer which forms the fiber surface, wherein the nonwoven structure is characterized by droplets of water containing 0.9 wt. % NaCl (“saline” or “salinated water”) having a contact angle on the nonwoven structure, as determined by the method described herein of equal to or less than 90° following at least 3 insults of salinated water. All individual values and subranges from equal to or less than 90° are disclosed herein and included herein. For example, following at least 3 insults of salinated water, the contact angle can range from an upper limit of 90°, or in the alternative, from an upper limit of 80°, or in the alternative, from an upper limit of 70°, or in the alternative, from an upper limit of 60°.

In yet another alternative embodiment, the present disclosure further provides an article comprising the nonwoven structure according to any embodiment disclosed herein.

In an alternative embodiment, the present disclosure provides a process for making a hydrophilic nonwoven structure, nonwoven structures and articles containing the nonwoven structures, in accordance with any of the preceding embodiments, except that the substance is allyl alcohol or hydroxyl ethyl acrylate.

In an alternative embodiment, the present disclosure provides a process for making a hydrophilic nonwoven structure, nonwoven structures and articles containing the nonwoven structures, in accordance with any of the preceding embodiments, except that the inert gas comprises nitrogen, helium, argon or combinations thereof.

In an alternative embodiment, the present disclosure provides a process for making a hydrophilic nonwoven structure, nonwoven structures and articles containing the nonwoven structures, in accordance with any of the preceding embodiments, except that the fiber is selected from the group consisting of polypropylene homopolymer (hPP) monocomponent fibers, random copolymer polypropylene fibers, polyethylene monocomponent fibers, styrenic block copolymer monocomponent fibers, bicomponent fibers having a sheath made from polyethylene and a core which comprises one or more selected from the group consisting of polyester, polyamide, styrene block copolymers, and polyolefins (including PP and elastomeric materials). The fiber may comprise any combination of two or more fibers as described herein. For example, the fiber may include both hPP monocomponent fibers and polyethylene sheath and polyester core fibers.

In an alternative embodiment, the present disclosure provides a process for making a hydrophilic nonwoven structure, nonwoven structures and articles containing the nonwoven structures, in accordance with any of the preceding embodiments, except that the exposing the nonwoven structure to the atmospheric plasma does not alter the internal structure of the nonwoven structure.

In an alternative embodiment, the present disclosure provides a process for making a hydrophilic nonwoven structure, nonwoven structures and articles containing the nonwoven structures, in accordance with any of the preceding embodiments, except that the exposing the nonwoven structure to the atmospheric plasma does not alter the internal structure of the fibers.

In an alternative embodiment, the present disclosure provides a process for making a hydrophilic nonwoven structure, nonwoven structures and articles containing the nonwoven structures, in accordance with any of the preceding embodiments, except that the hydrophilic moiety is selected from the group consisting of hydroxyl groups and carboxylic acid groups.

In an alternative embodiment, the present disclosure provides a process for making a hydrophilic nonwoven structure, nonwoven structures and articles containing the nonwoven structures, in accordance with any of the preceding embodiments, except that the structure further exhibits a surface energy equal to or greater than 40 dynes/cm. All individual values and subranges from equal to or greater than 40 dynes/cm are included herein and disclosed herein. For example, the nonwoven structure can have a surface energy equal to or greater than 40 dynes/cm, or in the alternative, the nonwoven structure can have a surface energy equal to or greater than 42 dynes/cm, or in the alternative, the nonwoven structure can have a surface energy equal to or greater than 44 dynes/cm, or in the alternative, the nonwoven structure can have a surface energy equal to or greater than 46 dynes/cm, or in the alternative, the nonwoven structure can have a surface energy equal to or greater than 48 dynes/cm.

In an alternative embodiment, the present disclosure provides a process for making a hydrophilic nonwoven structure, nonwoven structures and articles containing the nonwoven structures, in accordance with any of the preceding embodiments, except that the article is an absorbent article selected from the group consisting of diapers, adult incontinence products, training pant, feminine hygiene pads, and panty liners.

In an alternative embodiment, the present disclosure provides a process for making a hydrophilic nonwoven structure, nonwoven structures and articles containing the nonwoven structures, in accordance with any of the preceding embodiments, except that the article is disposable.

In an alternative embodiment, the present disclosure provides a process for making a hydrophilic nonwoven structure, nonwoven structures and articles containing the nonwoven structures, in accordance with any of the preceding embodiments, except that the article comprises a topsheet having an upper surface, a backsheet and a core disposed between the topsheet and the backsheet, wherein the upper surface of the topsheet comprises the nonwoven structure according to any embodiment disclosed herein.

### Examples

The following examples illustrate the disclosed subject matter but are not intended to limit the scope of the disclosed subject matter.

### Materials

Three types of nonwoven materials were used to prepare the inventive and comparative examples: (1) bicomponent spunbond (polypropylene/polyethylene, 50/50 (PP/PE)); (2) polypropylene homopolymer (hPP) monocomponent spunbond; and (3) soft polypropylene monocomponent spunbond nonwovens, as described in U.S Application Publication Nos. 2013/0237111, 2012/0045956, and 2012/0046400, were treated with dielectric barrier discharge atmospheric plasma. U.S Application Publication Nos. 2013/0237111, 2012/0045956, and 2012/0046400 are incorporated herein in its entirety by reference. Each of the nonwoven substances had a basis weight of 20 grams per square meter. Two substances were examined to modify the nonwoven materials applying —OH functionality: (1) hydroxyl ethyl acrylate monomer and (2) allyl alcohol monomer.

### Plasma Equipment

A PLASMAZONE Atmospheric Plasma system at VITO—Flemish Institute for Technological Research (Mol, Belgium) was used. The PLASMAZONE system is similar to commercially available atmospheric plasma systems such as the system from SOFTAL 3DT LLC (Germantown, Wis.), except that the PLASMAZONE is capable of introducing atomized liquid precursor into the plasma. This system generates a plasma using dielectric barrier discharge. The plasma, i.e. a non-thermal discharge (low temperature), is generated by the application of high voltages across a small gap wherein a non-conducting coating prevents the transition of the plasma discharge into an arc. In summary, the PLASMAZONE includes an upper electrode connected to high voltage and a lower electrode being grounded. A Dielectric Barrier Discharge is generated between the electrodes in a N₂ atmosphere. The gas mixtures introduced in the system can be chosen in such way that desired functionalities can be introduced onto the surface of the substrate. Typical gases used are N₂, H₂, CO₂, and NH₃.

Plasma Treatment of Nonwoven Materials

A4 size samples of each nonwoven material were plasma-treated under nitrogen atmosphere in the presence of one of the two —OH functionality substances to provide Inventive Examples 1-6, as shown in Table 1.

TABLE 1

| | | | Line | Treatment |
|---|---|---|---|---|
| Inv. | | —OH Functionality | Speed | Time |
| Ex. | Substrate | Substance | (m/min) | (seconds) |
| 1 | Bicomponent | allyl alcohol | 5 | 17 |
| | Spunbond (PP/PE) | monomer | | |
| 2 | hPP mono- | allyl alcohol | 5 | 17 |
| | component Spunbond | monomer | | |
| 3 | Soft PP mono- | allyl alcohol | 5 | 17 |
| | component spunbond | monomer | | |
| 4 | Bicomponent | hydroxyethyl | 4 | 22 |
| | Spunbond (PP/PE) | acrylate monomer | | |
| 5 | hPP mono- | hydroxyethyl | 4 | 22 |
| | component Spunbond | acrylate monomer | | |
| 6 | Soft PP mono- | hydroxyethyl | 4 | 22 |
| | component spunbond | acrylate monomer | | |

Comparative Example 1 was an untreated bicomponent spunbond (PP/PE). Comparative Example 2 was an untreated hPP mono-component spunbond. Comparative Example 3 was a soft PP monocomponent spunbound.

Table 2 provides the surface energy results for each of the Inventive and Comparative Examples. Each of Inventive Examples 1 and 4-6 exhibited a surface energy of at least 54 dynes/cm (the maximum surface energy ink used in testing). Inventive Example 2 had a surface energy of 44 dynes/cm and Inventive Example 3 had a surface energy of 42 dynes/cm. Each of Comparative Examples 1-3 exhibited a surface energy of 34 dynes/cm.

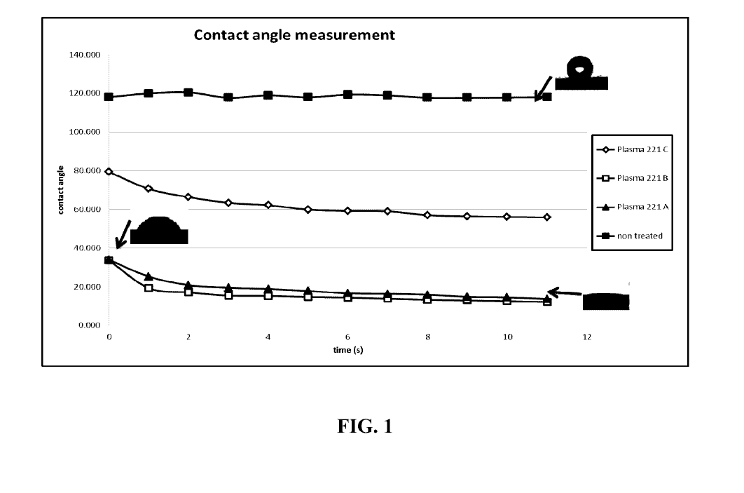

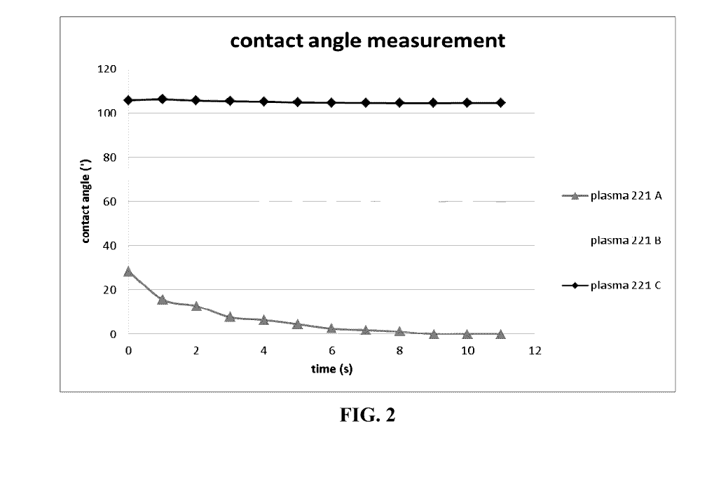

FIGS. 1-2 illustrate the contact angle of droplets of saline solution (0.9 wt % NaCl in water) for each of Inventive Examples 4-6 for measurements made following plasma treatment (for the Inventive Examples) and aged 6 weeks following plasma treatment, respectively. FIG. 1 also shows the initial contact angle for Comparative Example 1. As can be seen, the bicomponent spunbound (PP/PE) plasma-treated with hydroxyethyl acrylate monomer consistently provided the lowest contact angle.

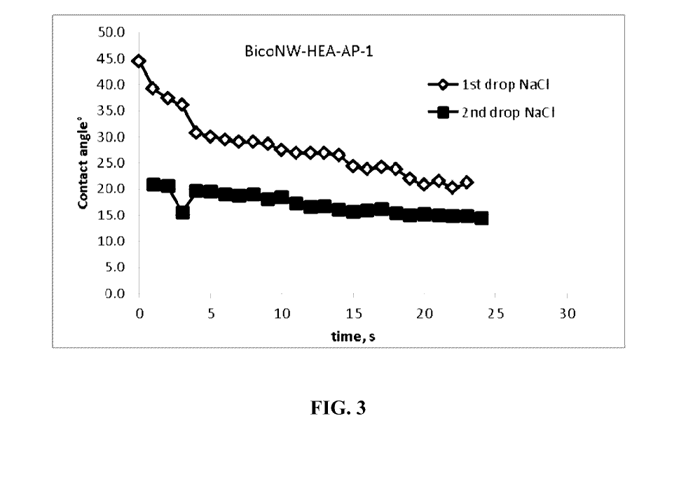

FIG. 3 illustrates the contact angle of droplets of saline (0.9 wt % NaCl in water) following a single insult and following a second insult for bicomponent spunbound plasma-treated with hydroxyethyl acrylate monomer. As can be seen in FIG. 3, the plasma-treated bicomponent spunbound maintained hydrophilicity following repeated saline insult.

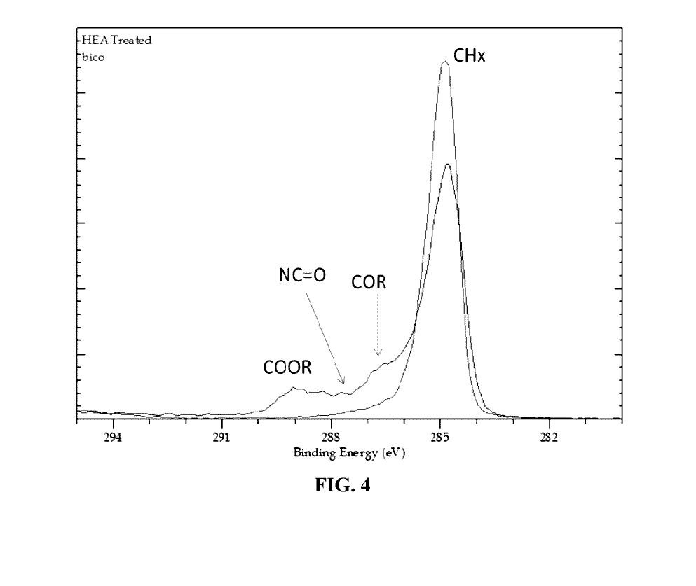

The surface compositions from the first 10 nm of Inventive Example 4 and Comparative Example 1 are listed in Table 2. Both sides of Inventive Example 4 were analyzed. The increased oxygen and nitrogen at the surfaces of Inventive Example 4 indicate surface modification. The carbon spectrum of Inventive Example 4 indicates an ester type carbon (—COOR) while the carbon spectrum of Comparative Example 1 shows —(CH)x only (See FIG. 4). Surface compositions neglect trace impurities and hydrogen.

TABLE 2

| Example | Carbon (wt %) | Nitrogen (wt %) | Oxygen (wt %) |
|---|---|---|---|
| Inv. Ex. 4, side A | 73.8 | 4.9 | 20 |
| Inv. Ex. 4, side B | 78.4 | 5 | 15.3 |
| Comp. Ex. 1 | 97.9 | None detected | 1.4 |

Fiber surface of Comparative Example 1 and Inventive Example 4 were analyzed using scanning electron microscope (SEM) with secondary electron contrast. Apparent fiber surface morphology did not reveal any morphology change or fiber breakage.

### Test Methods

Test methods include the following:

### Surface Energy Measurements

To evaluate the surface energy of the plasma-treated samples, test inks were used. Small droplets of the test inks were applied on the surface of the nonwoven material. The immersion of the test ink in the material was evaluated. The surface energy range of the test inks was from 34 to 54 dynes/cm. Surface energy is measured using ARCOTEC test inks and test pens available from Lotar Enterprises. As a starting point for each check a test ink or test pen with a medium value should be applied, e.g., 38 mN/m (dyne/cm). If the line of ink stays unchanged for at least 2 seconds on the surface of the material without turning into droplets, the surface energy of the material is the same or higher than the surface tension of the fluid. In this case, the test ink/test pen with the next higher value is applied to the surface, e.g., 40 mN/m (dyne/cm). This check has to be repeated with the next higher value of surface tension up to the point, at which within 2 seconds the line of fluid turns into separate droplets. If already at the starting point (38 mN/m (dyne/cm)) droplets are formed from the line of fluid, the check is continued with test inks/test pens of lower values, which is often the case with metals. As a general limit often 32 mN/m (dyne/cm) are mentioned: If the surface energy level is below this value, the adhesion will be poor, above this value the adhesion will be good or sufficient.

### XPS

X-ray photoelectron spectroscopy (XPS) measurements were performed using a Thermo K-alpha XPS instrument with a standard Monochromatic Al Ka 72 Watts (12 kV, 6 mA) X-ray source. Peak areas were evaluated using the instrument specific relative sensitivity factors.

### SEM

For SEM study, the samples were coated on both sides with Cu (copper) for 150 s at 80 mA (High Resolution Sputter Coater 208 HR, Cressington). The SEM images of sample surface were obtained with NOVA nanoSEM 600 (FEI, Eindhoven, The Netherlands) operated at high vacuum mode with 5 kV and spot 3.5. The images were recorded using EDT secondary electron detector and vCD backscattered electron detector.

### Contact Angle

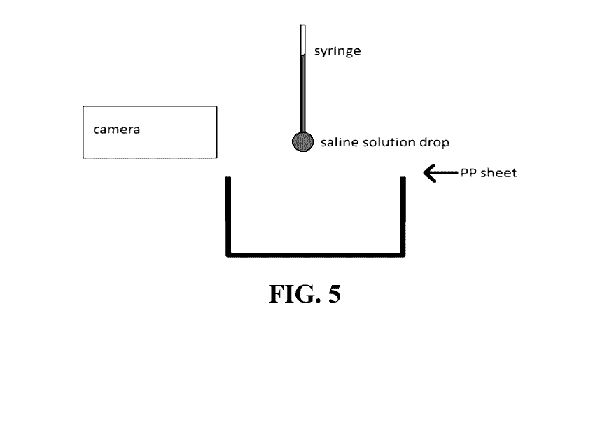

Prepare a few liters of saline solution (9 g/l of sodium chloride in water). Using the set up shown in FIG. 5, affix a sheet of the material being tested onto a hollow support with double-sided adhesive. Put the saline solution into the syringe suspended above the sheet. Apply a droplet of the saline solution to the sheet and when the droplet touches the sheet begin photographing the droplet at twelve frames at one second intervals. When the recording is finished, measure the contact angle of the droplet for each image.

The disclosed subject matter may be embodied in other forms without departing from the spirit and the essential attributes thereof, and, accordingly, reference should be made to the appended claims, rather than to the foregoing specification, as indicating the scope of the disclosed subject matter.

1. A process for making a hydrophilic nonwoven structure comprising:
a. forming a nonwoven structure comprising fibers; and
b. exposing the nonwoven structure to an atmospheric plasma comprising an inert gas and a substance having a polar group and which can be vaporized or made into an aerosol and which forms a free radical upon exposure to a dielectric barrier discharge.

2. The process of claim 1, wherein the substance is allyl alcohol or hydroxyl ethyl acrylate.

3. The process according to claim 1, wherein the inert gas comprises nitrogen, helium, argon or combinations thereof.

4. The process according to claim 1, wherein the fiber is selected from the group consisting of hPP monocomponent fibers, random copolymer PP fibers, polyethylene monocomponent fibers, styrenic block copolymer monocomponent fibers, bicomponent fibers having a sheath made from polyethylene and a core which comprises one or more selected from the group consisting of polyester, polyamide, styrene block copolymers, and polyolefins (including PP and elastomeric materials).

5. The process according to claim 1, wherein the exposing the nonwoven structure to the atmospheric plasma does not alter the internal structure of the nonwoven structure.

6. The process according to claim 1, wherein the exposing the nonwoven structure to the atmospheric plasma does not alter the internal structure of the fibers.

7. A hydrophilic nonwoven structure produced by the process according to claim 1.

8. A nonwoven structure comprising fibers having a chemically modified surface, wherein the chemically modified surface comprises a hydrophilic moiety covalently bonded to a polymer which forms a fiber surface, wherein the nonwoven structure is characterized by having a contact angle equal to or less than 90° following at least 3 insults of water containing 0.9 wt % NaCl.

9. The nonwoven structure according to claim 8, wherein the fibers are selected from the group consisting of hPP monocomponent fibers, random copolymer PP fibers, polyethylene monocomponent fibers, styrenic block copolymer monocomponent fibers, bicomponent fibers having a sheath made from polyethylene and a core which comprises one or more selected from the group consisting of polyester, polyamide, styrene block copolymers, and polyolefins (including PP and elastomeric materials).

10. The nonwoven structure according to claim 8, wherein the hydrophilic moiety is selected from the group consisting of hydroxyl groups and carboxylic acid groups.

11. The nonwoven structure according to claim 8, wherein the structure further exhibits a surface energy equal to or greater than 40 dynes/cm.

12. An article comprising the nonwoven structure according to claim 8.

13. The article according to claim 12, wherein the article is an absorbent article selected from the group consisting of diapers, adult incontinence products, training pant, feminine hygiene pads, and panty liners.

14. The article according to claim 13, wherein the article is disposable.

15. (canceled)
